(19) United States
(12) Patent Application Publication
(10) Pub. No.: US 2020/0188259 A1
(43) Pub. Date: Jun. 18, 2020

(54) TWO-COMPONENT HAIR CARE PRODUCT FOR PREPARING AN EMULSION FOR GROOMING HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Hamburg (DE)

(73) Assignee: Henkel AG & Co. KGaA, Duesseldorf (DE)

(21) Appl. No.: 16/716,076

(22) Filed: Dec. 16, 2019

(30) Foreign Application Priority Data
Dec. 18, 2018 (DE) 10 2018 222 141.3

(51) Int. Cl. A61K 8/41; A61K 8/34; A61K 8/31; A61K 8/42; A61Q 5/06

(52) U.S. Cl. CPC A61K 8/416; A61K 8/342; A61Q 5/06; A61K 8/42; A61K 8/31

(57) ABSTRACT

The present disclosure relates to a two-component hair care product which comprises an anhydrous carrier as a first component and an aqueous carrier as a second component separately from each other, wherein the anhydrous carrier contains a) a branched or unbranched alkane, b) a branched or unbranched fatty alcohol, and c) a monovalent branched or monovalent unbranched C2 to C8, preferably C4 to C6 alcohol. The present disclosure relates further to a method for applying the two-component hair care product and use thereof.

### CROSS-REFERENCE TO RELATED APPLICATION

This application claims priority to German Patent Application No. 10 2018 222 141.3, filed Dec. 18, 2018, which is incorporated herein by reference in its entirety.

### TECHNICAL FIELD

The present disclosure relates to the field of preparing hair care products in cosmetics.

### BACKGROUND

The present disclosure relates particularly to a two-component hair care product which comprises an anhydrous carrier as a first component and an aqueous carrier as a second component separately from each other, wherein the anhydrous carrier contains a) a branched or unbranched alkane, b) a branched or unbranched fatty alcohol and c) a monovalent branched or monovalent unbranched C2 to C8, preferably C4 to C6 alcohol. The present disclosure relates further to a method for applying the two-component hair care product and use thereof.

### BRIEF SUMMARY

Cosmetic products for cleaning and/or grooming hair have been known for a long time and are regularly improved and/or adapted to the changing needs of consumers. For example, consumers expect that a modern hair grooming product or conditioner will clean and smooth their hair, make it easier to comb, soft, shiny, easy to style, as well as moisturising, combating dandruff and lending it more volume, and more. Many active substances are known which can be used in such a cosmetic product to satisfy one or more of the requirements listed above. However, the presence of large quantities of different substances can destabilise the hair cosmetic, and for this reason manufacturers usually attempt to satisfy as many of the aforementioned requirements as possible using a small number of compatible substances.

A large number of different components may have the effect of limiting freedom of formulation during the development of cosmetic products, because components interact with each other or interfere with the structure. For example, substances may upset the desired structure of an emulsion, although it is precisely the desired structure of the emulsion that may be associated with advantageous cosmetic effects. Components of an aqueous phase and components of a fatty phase may also have properties that hinder the formation of emulsions. Thus even the emulsion formation, that is to say the preparation of the emulsion, has an effect on the formulation. The freedom of formulation is further limited by the requirement to ensure good biodegradability. The task of complying with the totality of all requirements to a satisfactory degree presents developers with considerable challenges.

The problem addressed by the present disclosure consists in providing a cosmetic product, particularly a hair grooming and/or conditioning agent constituting an emulsion that can be prepared easily. The problem addressed by the present disclosure consisted more particularly in providing a cosmetic product with the capability to offer the maximum possible freedom of formulation. A further problem addressed by the present disclosure consisted in providing a cosmetic product that can be managed with ease by consumers.

The problem is solved with the object of claim 1. Accordingly, a first object of the present disclosure is a two-component hair care product comprising separately from each other an anhydrous carrier as a first component and an aqueous carrier as a second component, anhydrous carrier comprises a) a branched or unbranched alkane having a chain length from C10 to C30, preferably from C12 to C24, more preferably from C14 to C20, b) a branched or unbranched fatty alcohol having a chain length from C10 to C22, preferably C10 to C18, more preferably from C12 to C16, and c) a monovalent branched or a monovalent unbranched C2 to C8, preferably C4 to C6 alcohol.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

According to a preferred embodiment of the present disclosure, the aqueous carrier or the anhydrous carrier contains a cationic or non-ionic or anionic tenside which comprises one or more of the following compounds: d) an alkyl trimonium compound having a chain length in the alkyl group from C8 to C22, preferably from C10 to C18, more preferably from C12 to C16 and a physiologically or cosmetically acceptable anion which is preferably a halide, particularly a chloride or bromide, or a methosulfate, e) a fatty alcohol propoxylate having an alkyl chain length from C6 to C30, preferably from C8 to C22, more preferably C10 to C18, wherein the fatty alcohol propoxylate comprises from about 1 to about 15, preferably from about 2 to about 10, more preferably from about 3 to about 6 propylene oxide groups, f) a fatty alcohol sulfate with an alkyl chain length from C8 to C22, preferably from C10 to C20, more preferably C12 to C18, and a physiologically or cosmetically acceptable cation which is preferably sodium, magnesium or ammonium.

Surprisingly, it was found that the two components, i.e. the aqueous carrier and the anhydrous carrier, are readily mixable with each other to form an emulsion. A number of advantageous effects are associated with this. On the one hand, constituents may be present in the differing components which—if they are present in the same composition—interact in such a way that over a prolonged period they present a risk of unfavourable effects. This therefore increases the freedom of formulation, since constituents in the components do not come into contact with each other until shortly before application. The fact that constituents of the components are incompatible over a prolonged period is only a minor consideration. On the other hand, with the object as contemplated herein users have at their disposal the capability of using a stable emulsion: Because of the ease with which the aqueous carrier and the anhydrous carrier can be combined to yield an emulsion, the user can mix the components shortly before the application and immediately obtain an emulsion which is stable until it is applied. Moreover, the user is also in possession of an experience product. It may be used to create an application-ready cosmetic product according to the user's needs. A two-component kit enhances confidence in the performance capability of the product.

The two-component hair care product is preferably not a hair dye or bleaching agent. Apart from the points discussed previously, it is preferable if the two-component hair care product as contemplated herein does not contain any oxidising agents, no direct dyes and no oxidation dye precursors for dyeing keratinous fibres, particularly human hair.

The first carrier of the composition as contemplated herein should be anhydrous, more particularly substantially anhydrous. The person skilled in the art understands an anhydrous carrier to be a composition to which no water is added. Given the fact that raw materials generally are not entirely water-free, the anhydrous carrier contains a small quantity of water. Moreover, products always absorb a certain degree of moisture. A preferred anhydrous carrier is substantially free of water. More preferably, the anhydrous carrier comprises from 0 to about 3 wt % water, more preferably from about 0.1 to about 2 wt %, most preferably from about 0.2 to about 1 wt % relative to the total weight of the carrier which is described as anhydrous.

As a first constituent, the anhydrous carrier of the two-component hair care product as contemplated herein comprises a branched or unbranched alkane. This is essentially selected such that it is fluid at a temperature of about 20° C. and a pressure of about 10⁵ Pa. Consequently, the alkanes with a higher number of carbon atoms tend to be branched alkanes, whereas the alkanes with a lower number of carbon atoms may be linear or branched alkanes. The alkane essentially constitutes the organic basis of the anhydrous carrier.

Preferred alkanes are thus liquid paraffin oil, isoparaffinic oil or synthetic hydrocarbons. One product that is available commercially may be 1,3-di-(2-ethyl-hexyl)-cyclohexane (Cetiol® S). Most particularly preferred is the alkane dodecane or the isoalkane isododecane.

As a second constituent, the anhydrous carrier of the two-component hair care product as contemplated herein comprises a fatty alcohol. Fatty alcohols that are suitable for use are monovalent—that is to say fatty alcohols with one or two OH groups—saturated, mono- or polyunsaturated, branched or linear fatty alcohols. Fatty alcohols that are usable for the purposes of the present disclosure include for example decanol, dodecenol, decenol, octadienol, dodecadienol, decadienol, oleyl alcohol, erucyl alcohol, ricinol alcohol, lauryl alcohol, myristyl alcohol, cetearyl alcohol, capric alcohol, linoleyl alcohol and linolenyl alcohol. Particularly preferred are cetearyl alcohol and myristyl alcohol. Particularly preferred fatty alcohols are also Coconut Alcohol (INCI), Cetearyl Alcohol (INCI), Cetyl Alcohol (INCI), Stearyl Alcohol (INCI), Myristyl Alcohol (INCI), Lauryl Alcohol (INCI) and Decyl Alcohol (INCI). Fatty alcohol blends representing a mixture of various fatty alcohols are also usable as contemplated herein.

As a third component, the anhydrous carrier of the two-component hair care product as contemplated herein contains a monovalent linear or branched C2-C6 alcohol. Preferred alcohols include ethanol, propanol, isopropanol, 1,2-propylene glycol, butanol, isobutanol, tert.-butanol, n-pentanol, iso-pentanols, particularly 3-methyl-1-butanol, n-hexanol, iso-hexanols, ethylene glycol, glycerin, 1,2-Pentanediol, 1,5-Pentanediol, 2-Methyl-2,4-pentanediol, 1,2-Hexanediol or 1,6-Hexanediol.

According to a preferred embodiment of the present disclosure, the cationic tenside is a tetraalkylammonium compound, preferably a trimethyl alkylammonium halide, preferably cetrimonium chloride, or a trimethyl alkylammonium methosulfate, or a N-[3-(Dimethylamino)propyl]alkanamide, particularly N-[3-(Dimethylamino)propyl]octadecanamide, or stearamidopropyl dimethylamine

Most particularly preferably, the aqueous carrier contains Cetrimonium Chloride (INCI) and/or Stearamidopropyl Dimethylamine (INCI).

According to the preferred embodiment, the aqueous carrier contains as a constituent a cationic tenside having formula (1) which represents a tetraalkylammonium compound, that is to say

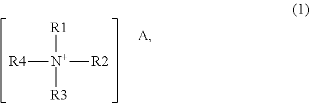

in which R1, R2, R3 and R4 each independently of each other stand for hydrogen, a methyl group, a phenyl group, a benzyl group, a saturated, branched or unbranched alkyl radical with a chain length from 12 to 30 carbon atoms, which may or may not be substituted with one or more hydroxy groups, providing that at least one of the radicals R1, R2, R3 or R4 is not hydrogen and represents a saturated, branched or unbranched alkyl radical with a chain length from 12 to 30 carbon atoms, and wherein A stands for a physiologically compatible anion, for example halides such as chloride or bromide and methosulfate.

According to a preferred embodiment of the present disclosure, the cationic tenside with formula (1) selected from the group including lauryl trimethylammonium chloride, lauryl trimethylammonium methosulfate, cetyl trimethylammonium chloride, cetyl trimethylammonium bromide, cetyl trimethylammonium methosulfate, dicetyl dimethylammonium chloride, dicetyl dimethylammonium methosulfate, tricetyl methylammonium chloride, tricetyl methylammonium methosulfate, stearyl trimethylammonium chloride, stearyl trimethylammonium methosulfate, distearyl dimethylammonium chloride, distearyl dimethylammonium methosulfate, lauryl dimethylbenzylammonium chloride, behenyl trimethylammonium chloride, behenyl trimethylammonium bromide, behenyl trimethylammonium methosulfate, dimethyl dibehenylammonium chloride, tribehenyl methyltrimonium chloride, cocotrimonium chloride, cocotrimonium methosulfate, dicocodimonium chloride, dicocodimonium methosulfate, tricocomonium chloride, tricocomonium methosulfate and mixtures thereof.

Further particularly preferred cationic tensides which may be used as contemplated herein are selected from esterquats. Particularly preferred esterquats as contemplated herein are distearoylethyl hydroxyethylmonium methosulfate and dicocoylethyl hydroxyethylmonium methosulfate. Such esterquat tensides are marketed commercially under the trade names Rewoquat®, Stepantex®, Dehyquart® and Armocare®, for example. The products Quartamin BTC 131, Armocare® VGH-70, a N,N-Bis(2-palmitoyloxyethyl)dimethylammonium chloride, and Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such esterquats.

As an alternative to constituent d), the aqueous or anhydrous carrier contains constituent e), specifically a fatty alcohol propoxylate with an alkyl chain length from C6 to C30, preferably from C8 to C22, more preferably C10 to C18, wherein the fatty alcohol propoxylate comprises from about 1 to about 15, preferably from about 2 to about 10, more preferably from about 3 to about 6 propylene oxide groups.

As a most particularly preferred fatty alcohol propoxylate, the anhydrous carrier contains PPG-3-Caprylyl Ether (INCI), a caprylyl ether that contains three propylene glycol groups.

As a further alternative to constituent d), the aqueous or anhydrous carrier may contain constituent f) specifically a fatty alcohol sulfate with an alkyl chain length from C8 to C22, preferably from C10 to C20, more preferably C12 to C18, and a physiologically or cosmetically acceptable cation which is preferably sodium, magnesium or ammonium.

The counterions in the cationic tensides are not a significant consideration in the solution of the problem addressed by the present disclosure. In each case, the anion may be a physiologically or cosmetically acceptable anion, preferably a halide, particularly chloride or bromide, or a methosulfate.

Similarly, the counterions in the anionic tensides are not a significant consideration in the solution of the problem addressed by the present disclosure. In each case, the cation may be a physiologically or cosmetically acceptable cation, preferably sodium, magnesium or ammonium, particularly sodium.

A preferred two-component hair care product contains in the first component the branched or unbranched alkane in a quantity from about 1 to about 50 wt %, preferably from about 2 to about 45 wt %, more preferably from about 3 to about 40 wt %, most preferably from about 4 to about 35 wt % relative to the total weight of the first component, and/or the two-component hair care product contains the fatty alcohol in a quantity from about 5 to about 50 wt %, preferably from about 6 to about 45 wt %, more preferably from about 7 to about 40 wt %, most preferably from about 8 to about 35 wt % relative to the total weight of the first component, and/or the two-component hair care product contains the monovalent linear or the monovalent branched alcohol in a quantity from about 4 to about 50 wt %, from about 6 to about 45 wt %, from about 8 to about 40 wt %, from about 10 to about 30 wt % relative to the total weight of the first component.

A further preferred two-component hair care product contains in the first component or in the second component the alkyl trimonium compound in a quantity from about 0.1 to about 5 wt %, preferably from about 0.5 to about 4 wt %, more preferably from about 1 to about 3 wt %, and/or the fatty alcohol propoxylate in a quantity from about 0.1 to about 10 wt %, preferably from about 0.25 to about 7.5 wt %, more preferably from about 0.5 to about 5 wt % relative to the total weight of the first and/or second component. In each case, quantities indicated refer to the component in which the constituent in question is contained.

The ranges indicated are associated with the advantage that the solution to the abovementioned problem addressed by the present disclosure can be attained.

Preferred two-component hair care products contain the first component and the second component in a weight ratio of the first component to the second component in the range from about 10 to 1 to about 1 to 10, preferably from about 5 to 1 to about 1 to 5, more preferably from about 2 to 1 to about 1 to 2. Particularly when the components contain the constituents in the preferred quantities specified above, two-component hair care products may be prepared that are easy to dispense in metered quantities, since the abovementioned weight ratios according to this preferred embodiment are easily manageable.

In a preferred two-component hair care product, the first component is a transparent composition, and/or the second component is a transparent composition, preferably both the first and second components are transparent compositions. This preferred embodiment responds to a more demanding customer requirement.

The products as contemplated herein further contain preferably at least one silicon polymer selected from the group of dimethiconols and/or the group of aminofunctional silicones and/or the group of dimethicones and/or the group of cyclomethicones. However, for each of these groups, arylated silicones of the respective groups of silicones are not included in the compounds as contemplated herein.

The dimethicones as contemplated herein may be linear or branched or cyclic or also cyclic and branched. The dimethicones are contained in the compositions as contemplated herein in quantities from about 0.01 to about 10 wt %, preferably from about 0.01 to about 8 wt %, particularly preferably from about 0.1 to about 7.5 wt % and especially from about 0.1 to about 5 wt % relative to the total composition. Hair treatment products that are preferred as contemplated herein contain from about 0.01 to about 10 wt %, preferably from about 0.01 to about 8 wt %, particularly preferably from about 0.1 to about 7.5 wt % and especially from about 0.2 to about 5 wt % aminofunctional silicone(s) and/or diquaternary silicone relative to its weight. The cyclic dimethicones described as Cyclomethicones according to INCI are also usable as contemplated herein to good effect.

A central aspect of the present disclosure relates to the feature according to which the hair care product as contemplated herein is provided in the form of a two-component hair care product. The two-component hair care product is prepared in such manner that the user can take both components out of an associated container. According to a preferred embodiment of the present disclosure, the first component and the second component are provided in such manner that they are separated from each other by a barrier which is substantially impermeable to liquids, in particular by at least one film. This may be designed in such manner that two adjoined sachets each contain one component. According to a preferred embodiment of the present disclosure, the first component and the second component are contained in a packaging unit in such manner that they are separated from each other by a barrier which is substantially impermeable to liquids, in particular by at least one film. The barrier which is impermeable to liquids is designed to ensure that the two components do not mix with one another until the packaging unit is opened.

The problem to be addressed by the present disclosure is further solved by the object of claim 12. A second object of the present disclosure is therefore a method for treating hair, wherein in a first step the first component and the second component of the two-component hair care product as contemplated herein are mixed so that an emulsion is formed, in a second step the emulsion is allowed to stand for a period between about 1 second and about 30 minutes, preferably between about 20 seconds and about 20 minutes, more preferably between about 30 seconds and about 10 minutes, and in a third step following on from the second step the emulsion is applied to the keratinous fibres.

A major advantage of the present disclosure consists in the ease with which an emulsion may be formed with the aqueous carrier and the anhydrous carrier. An emulsion may be obtained quite quickly by agitation. This emulsion is then ready for use immediately and is spread on the keratinous fibres, in particular the hair in accordance with the method for application as contemplated herein.

According to a preferred embodiment of the method as contemplated herein, the emulsion applied to the keratinous fibres is left on the keratinous fibres. According to this first variant, the product as contemplated herein is used as a leave-on product. Alternatively, according to another preferred embodiment the emulsion applied to the keratinous fibres is rinsed out of the keratinous fibres after a period of from about 20 seconds to about 5 minutes, preferably from about 45 seconds to about 2 minutes. According to this alternative, the composition as contemplated herein is used as a rinse-off product.

According to a further preferred embodiment of the present disclosure, in the first step the first component and the second component are mixed with each other at a temperature of from about 15 to about 30° C. The preparation of the emulsion is thus preferably carried out without a heating step. By virtue of the inventive formulation of the aqueous carrier and the anhydrous carrier, said carriers may be mixed to form an emulsion without difficulty.

The problem to be addressed by the present disclosure is further solved by the object of claim 15. A third object of the present disclosure is thus the use of the two-component hair care product as contemplated herein for grooming, preferably conditioning keratinous fibres, in particular human hair.

Features relating to preferred embodiments of the first object of the present disclosure which are described only with reference thereto above naturally constitute corresponding features of preferred embodiments of the second object and the third object as well.

Most particularly preferred compositions as contemplated herein comprise at least one of the following embodiments A) to G):

### A)

Two-component hair care product, comprising an anhydrous carrier as a first component and an aqueous carrier as a second component, separated from each other, wherein the anhydrous carrier contains
a) a branched or unbranched alkane having a chain length from C10 to C30, preferably from C12 to C24, more preferably from C14 to C20,
b) a branched or unbranched fatty alcohol having a chain length from C10 to C22, preferably C10 to C18, more preferably from C12 to C16, and
c) a monovalent branched or a monovalent unbranched C2 to C8, preferably C4 to C6 alcohol,
and wherein the aqueous carrier or the anhydrous carrier contains a cationic or non-ionic or anionic tenside which comprises one or more of the following compounds:
d) an alkyl trimonium compound having a chain length in the alkyl group from C8 to C22, preferably from C10 to C18, more preferably from C12 to C16 and a physiologically or cosmetically acceptable anion which is preferably a halide, particularly a chloride or bromide, or a methosulfate,
e) a fatty alcohol propoxylate having an alkyl chain length from C6 to C30, preferably from C8 to C22, more preferably C10 to C18, wherein the fatty alcohol propoxylate comprises from about 1 to about 15, preferably from about 2 to about 10, more preferably from about 3 to about 6 propylene oxide groups,
f) a fatty alcohol sulfate with an alkyl chain length from C8 to C22, preferably from C10 to C20, more preferably C12 to C18, and a physiologically or cosmetically acceptable cation which is preferably sodium, magnesium or ammonium.

### B)

Two-component hair care product, comprising an anhydrous carrier as a first component and an aqueous carrier as a second component, separated from each other, wherein the anhydrous carrier contains
a) a branched or unbranched alkane having a chain length from C10 to C30, preferably from C12 to C24, more preferably from C14 to C20,
b) a branched or unbranched fatty alcohol having a chain length from C10 to C22, preferably C10 to C18, more preferably from C12 to C16, and
c) a monovalent branched or a monovalent unbranched C2 to C8, preferably C4 to C6 alcohol,
and wherein the aqueous carrier contains a cationic or non-ionic tenside which comprises one or more of the following compounds:
d) an alkyl trimonium compound having a chain length in the alkyl group from C8 to C22, preferably from C10 to C18, more preferably from C12 to C16 and a physiologically or cosmetically acceptable anion which is preferably a halide, particularly a chloride or bromide, or a methosulfate,
e) a fatty alcohol propoxylate having an alkyl chain length from C6 to C30, preferably from C8 to C22, more preferably C10 to C18, wherein the fatty alcohol propoxylate comprises from about 1 to about 15, preferably from about 2 to about 10, more preferably from about 3 to about 6 propylene oxide groups,
f) a fatty alcohol sulfate with an alkyl chain length from C8 to C22, preferably from C10 to C20, more preferably C12 to C18, and a physiologically or cosmetically acceptable cation which is preferably sodium, magnesium or ammonium.

### C)

Two-component hair care product, comprising an anhydrous carrier as a first component and an aqueous carrier as a second component, separated from each other, wherein the anhydrous carrier contains
a) a branched or unbranched alkane having a chain length from C10 to C30, preferably from C12 to C24, more preferably from C14 to C20,
b) a branched or unbranched fatty alcohol having a chain length from C10 to C22, preferably C10 to C18, more preferably from C12 to C16, and
c) a monovalent branched or a monovalent unbranched C2 to C8, preferably C4 to C6 alcohol,
and wherein the aqueous carrier contains a cationic tenside which is a tetraalkylammonium compound, preferably a trimethyl alkylammonium halide, a trimethyl alkylammonium methosulfate, a N-[3-(Dimethylamino)propyl]alkanamide, particularly N-[3-(Dimethylamino)propyl]octadecanamide, or stearamidopropyl dimethyl amine or cetrimonium chloride.

### D)

Two-component hair care product, comprising an anhydrous carrier as a first component and an aqueous carrier as a second component, separated from each other, wherein the anhydrous carrier contains
a) a dodecane or isododecane,
b) a branched or unbranched fatty alcohol with a chain length from C10 to C22, preferably C10 to C18, more preferably from C12 to C16, and
c) a monovalent branched or monovalent unbranched C2 bis C8, preferably C4 to C6 alcohol,
and wherein the aqueous carrier contains a cationic or non-ionic tenside which comprises one or more of the following:
d) an alkyl trimonium compound having a chain length in the alkyl group from C8 to C22, preferably from C10 to C18, more preferably from C12 to C16 and a physiologically or cosmetically acceptable anion which is preferably a halide, particularly a chloride or bromide, or a methosulfate,
e) a fatty alcohol propoxylate having an alkyl chain length from C6 to C30, preferably from C8 to C22, more preferably C10 to C18, wherein the fatty alcohol propoxylate comprises from about 1 to about 15, preferably from about 2 to about 10, more preferably from about 3 to about 6 propylene oxide groups,
f) a fatty alcohol sulfate with an alkyl chain length from C8 to C22, preferably from C10 to C20, more preferably C12 to C18, and a physiologically or cosmetically acceptable cation which is preferably sodium, magnesium or ammonium.

### E)

Two-component hair care product, comprising an anhydrous carrier as a first component and an aqueous carrier as a second component, separated from each other, wherein the anhydrous carrier contains
a) a dodecane or isododecane,

### b) Coconut Alcohol (INCI), Cetearyl Alcohol (INCI), Cetyl Alcohol (INCI), Myristyl Alcohol (INCI), Lauryl Alcohol (INCI) and Decyl Alcohol (INCI), and

c) a monovalent branched or monovalent unbranched C2 bis C8, preferably C4 to C6 alcohol,
and wherein the aqueous carrier contains a cationic or non-ionic tenside which comprises one or more of the following:
d) an alkyl trimonium compound having a chain length in the alkyl group from C8 to C22, preferably from C10 to C18, more preferably from C12 to C16 and a physiologically or cosmetically acceptable anion which is preferably a halide, particularly a chloride or bromide, or a methosulfate,
e) a fatty alcohol propoxylate having an alkyl chain length from C6 to C30, preferably from C8 to C22, more preferably C10 to C18, wherein the fatty alcohol propoxylate comprises from 1 to 15, preferably from 2 to 10, more preferably from 3 to 6 propylene oxide groups,
f) a fatty alcohol sulfate with an alkyl chain length from C8 to C22, preferably from C10 to C20, more preferably C12 to C18, and a physiologically or cosmetically acceptable cation which is preferably sodium, magnesium or ammonium.

### F)

Two-component hair care product, comprising an anhydrous carrier as a first component and an aqueous carrier as a second component, separated from each other, wherein the anhydrous carrier contains
a) a dodecane or isododecane,

### b) Coconut Alcohol (INCI), Cetearyl Alcohol (INCI), Cetyl Alcohol (INCI), Myristyl Alcohol (INCI), Lauryl Alcohol (INCI) and Decyl Alcohol (INCI), and

c) a monovalent branched or monovalent unbranched C2 bis C8, preferably C4 to C6 alcohol, and
and wherein the aqueous carrier contains a cationic tenside which is a tetraalkylammonium compound, preferably a trimethyl alkylammonium halide, a trimethyl alkylammonium methosulfate, a N-[3-(Dimethylamino)propyl]alkanamide, particularly N-[3-(Dimethylamino)propyl]octadecanamide, or stearamidopropyl dimethyl amine or cetrimonium chloride.

### G)

Two-component hair care product, comprising an anhydrous carrier as a first component and an aqueous carrier as a second component, separated from each other, wherein the anhydrous carrier contains
a) a dodecane or isododecane,

### b) Coconut Alcohol (INCI), Cetearyl Alcohol (INCI), Cetyl Alcohol (INCI), Myristyl Alcohol (INCI), Lauryl Alcohol (INCI) and Decyl Alcohol (INCI), and

c) a monovalent branched or monovalent unbranched C2 bis C8, preferably C4 to C6 alcohol,
and wherein the aqueous carrier contains a non-ionic tenside which is a fatty alcohol propoxylate having an alkyl chain length from C6 to C30, preferably from C8 to C22, more preferably C10 bis C18, wherein the fatty alcohol propoxylate comprises from about 1 to about 15, preferably from about 2 to about 10, more preferably from about 3 to about 6 propylene oxide groups, wherein the fatty alcohol propoxylate is in particular PPG-3-Caprylyl Ether (INCI).

### EXAMPLES

The following formulations can be prepared:

### Example 1

Anhydrous phase

| Constituent | Content in wt % |
|---|---|
| Isododecane | 25.0 |
| Cetearyl alcohol | 15.0 |
| Myristyl alcohol | 20.0 |
| Ethanol | 23.6 |
| Dimethicone 5 cSt | 15.0 |
| Fragrance | 1.40 |

Aqueous phase

| Constituent | Content in wt % |
|---|---|
| Water | 95.5 |
| Sodium benzoate | 0.5 |
| Citric acid | 0.8 |
| Stearamidopropyl Dimethylamine (INCI) | 0.8 |
| Cetrimonium chloride | 2.4 |

The anhydrous phase of Example 1 was mixed with the aqueous phase. The two phases were mixed with each simply by shaking by hand and formed an emulsion.

The hair care product obtained in this way was applied to dry or damp hair, and rinsed out again with lukewarm water at 20° C. after an application time of 5 minutes. The hair treated in this way was soft and had a pleasing shine.

When the hair was subsequently dried, it was smooth and did not curl (no fizz).

In a variation of the hair treatment method as contemplated herein, the hair care product obtained in this way was sprayed onto the dry hair using a pump sprayer. The hair treated in this way was soft, had a pleasing shine, and did not curl (no frizz).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Two-component hair care product, comprising an anhydrous carrier as a first component and an aqueous carrier as a second component, separated from each other, wherein the anhydrous carrier comprises
a) a branched or unbranched alkane with a chain length from C10 to C30,
b) a branched or unbranched fatty alcohol with a chain length from C10 to C22, and
c) a monovalent branched or a monovalent unbranched C2 to C8.

2. Two-component hair care product according to claim 1, wherein the aqueous carrier or the anhydrous carrier comprises a cationic or non-ionic tenside which comprises one or more of the following:
d) an alkyl trimonium compound having a chain length in the alkyl group from C8 to C22 and a physiologically or cosmetically acceptable anion which is preferably a halide,
e) a fatty alcohol propoxylate having an alkyl chain length from C6 to C30, wherein the fatty alcohol propoxylate comprises from about 1 to about 15,
f) a fatty alcohol sulfate with an alkyl chain length from C8 to C22, and a physiologically or cosmetically acceptable cation which is preferably sodium, magnesium or ammonium.

3. Two-component hair care product according to claim 1, wherein the branched or unbranched alkane is dodecane or isododecane.

4. Two-component hair care product according to claim 1, wherein the branched or unbranched fatty alcohol is selected from the group of Coconut Alcohol (INCI), Cetearyl Alcohol (INCI), Cetyl Alcohol (INCI), Myristyl Alcohol (INCI), Lauryl Alcohol (INCI) and Decyl Alcohol (INCI).

5. Two-component hair care product according to claim 1, wherein the cationic tenside is a tetraalkylammonium compound, preferably a trimethyl alkylammonium halide, a trimethyl alkylammonium methosulfate, a N[3-(Dimethylamino)propyl]alkanamide.

6. Two-component hair care product according to claim 1, wherein the two-component hair care product comprises in the first component the branched or unbranched alkane in a quantity from about 1 to about 50 wt % relative to the total weight of the first component, comprises the fatty alcohol in a quantity from about 5 to about 50 wt %, relative to the total weight of the first component, and comprises the monovalent linear or the monovalent branched alcohol in a quantity from about 4 to about 50 wt %, from about 6 to about 45 wt %, from about 8 to about 40 wt %, from about 10 to about 30 wt % relative to the total weight of the first component.

7. Two-component hair care product according to claim 1, wherein the two-component hair care product comprises in the first component or the second component the alkyl trimonium compound in a quantity from about 0.1 to about 5 wt %, and or the fatty alcohol propoxylate in a quantity from about 0.1 to about 10 wt % relative to the total weight of the first and/or second component.

8. Two-component hair care product according to claim 1, wherein the weight ratio of the first component to the second component is from about 10 to 1 to about 1 to 10.

9. Two-component hair care product according to claim 1, wherein the first component is a transparent composition, and/or the second component is a transparent composition.

10. Two-component hair care product according to claim 1, wherein the first component and the second component are provided in such manner that they are separated from each other by a barrier which is substantially impermeable to liquids, or wherein the first component and the second component are included in a packaging unit in such manner that they are separated from each other by a barrier which is substantially impermeable to liquids.

11. Method for treating hair, wherein in a first step the first component and the second component of the two-component hair care product according to claim 1 are mixed with each other so that an emulsion is formed, in a second step the emulsion is allowed to stand for a period between about 1 second and about 30 minutes, and in a third step following on from the second step the emulsion is applied to the keratinous fibres.

12. Method according to claim 11, wherein the emulsion applied to the keratinous fibres is left on the keratinous fibres, or wherein the emulsion applied to the keratinous fibres is rinsed out of the keratinous fibres after a period of from about 20 seconds to about 5 minutes.

13. Method according to claim 11, wherein in the first step the first component and the second component are mixed with each other at a temperature of from about 15 to about 30° C.

14. Use of the two-component hair care product according to claim 1 to groom, preferably to condition, keratinous fibres.
